(19) United States
(12) Patent Application Publication
(10) Pub. No.: US 2005/0201948 A1
(43) Pub. Date: Sep. 15, 2005

(54) Excipient for use in dry powder inhalation preparations

(76) Inventors: Mark Jason Heath Ellison, West Sussex (GB); Theodora Antonia Maria Lambregts-Van Den Hurk, Veghel (NL)

Correspondence Address: THE WEBB LAW FIRM, P.C., 700 KOPPERS BUILDING, 436 SEVENTH AVENUE, PITTSBURGH, PA, 15219, US

(21) Appl. No.: 10/511,006

(22) PCT Filed: Apr. 12, 2002
(86) PCT No.: PCT/EP02/04207

§ 371 Date: May 9, 2005

(51) Int. Cl. A61L 9/04; A61K 9/14

(52) U.S. Cl. 424/46

(57) ABSTRACT

The present invention relates to an excipient for dry powder inhalation preparations comprising granules made of primary carrier material, which granules break down during inhalation in such a manner that they give a concentration of primary carrier material at stage 2 of the twin stage impinger determined by the antrone reaction of at least 5%.

The present invention relates to an excipient for use in dry powder inhalation preparations. The invention furthermore relates to dry powder inhalation preparations containing the excipient, to a method for making the excipient and to an excipient made of lactose.

The delivery of active molecules to the lungs can be achieved using metered dose inhalers (MDI), dry powder inhalers (DPI) or nebulisers. In the current market MDI are dominant with DPI a distant second and nebulisers further back. MDI have continued to be successful despite the difficulty of coordinating actuation with inhalation and the extensive deposition on the back of the oropharynx due to the high velocity of the droplets.

However, this success has been blighted in recent times by the environmental concerns over chlorofluorocarbons (CFCs), which have been used as propellants. The Montreal protocol in 1989 detailed the need to replace CFC propellants, because of their contribution to ozone depletion. This has resulted in the development of propellants which do not deplete ozone and an increase in activity in the DPI field.

There are a number of DPI products available on the market today, using many different technological approaches for delivering an active component to the lungs. To penetrate into the target areas of the lungs, active molecules must possess an aerodynamic particle size of less than 5 μm. This is achieved primarily by micronisation. The particles produced are, however, inherently cohesive/adhesive in nature due to an excess of surface free energy. The surface properties generated in manufacture can lead to adherence to the device and/or the formation of stable agglomerates, both of which can have a negative influence on the dose reproducibility as they are uncontrollable.

Therefore, traditionally a DPI product consists of the device, the active component and an inert carrier (i.e. excipient) with the purpose to aid flow and encourage dispersion. The active particles adhere to the surface of the carrier, ideally preventing segregation but allowing detachment during inhalation.

The preferred carrier material has always been α-lactose monohydrate. The reasons for this include the fulfillment of the carrier functions by improving flow, the availability of toxicological information and its relatively low price. The manipulation of lactose to balance the requirements of high and constant deposition values and good flow properties has focused primarily on the particle size distribution. However, a number of other techniques have been investigated to improve the performance of lactose as a carrier.

U.S. Pat. No. 5,254,330 describes the use of smooth crystals produced by controlled crystallization, which have a rugosity of less than 1.75.

An alternative to alpha-lactose monohydrate is Described in the International patent WO98/50015, which makes use of roller dried anhydrous lactose with a size between 50 and 250 μm and a rugosity between 1.9 and 2.4.

The lactose described in the prior art is in a crystalline form. The particle size is relatively small. It was found that the deposition of these known particles can be further improved.

It is known that decreasing the carrier particle size of a powder mixture, results in an increase in the fine particle fraction. As the particle size is reduced the relationship between the carrier lactose particle and micronised active component changes. For large carrier particles the active adheres to the surface of the carrier. As carrier size decreases and approaches that of the micronised active component the relationship is more of a weak agglomerate, which can be easily dispersed especially with the modern inhaler devices.

However, as the carrier particle size is decreased, so are the flow properties which affects the distribution of the active component within the mix and the dose reproducibility.

It is the object of the present invention to provide an excipient that can be used as a carrier in dry powder inhalation preparations and that consists of particles large enough to have suitable flow properties and a structure to promote dispersion.

This object is achieved by an excipient for dry powder inhalation preparations comprising granules made of primary carrier material, which granules break up during inhalation in such a manner that they give a concentration of primary carrier material on stage 2 of the twin stage impinger (e.g. by Erweka, UK) determined by the antrone reaction of at least 5%.

Preferably, the concentration of primary-carrier material at stage 2 of the twin stage impinger determined by the antrone reaction is at least 10%, more preferably at least 20%.

Such an excipient is obtainable by granulating a primary carrier material in a fluid binding agent, for example in a fluid bed dryer or a shear mixer, and drying the granules thus obtained. The fluid binding agent is preferably an aqueous solution of the primary carrier material. Alternatively, the fluid binding agent is a solvent, in particular ethanol. The properties of the excipient granules may be varied by choosing the fluid binding agent. A solvent will usually evaporate more quickly thus resulting in weaker granules that lead to a higher percentage at stage 2 of the twin stage impinger.

The strength of the granules can be manipulated by varying the process parameters such as the amount of fluid binding agent (granulation fluid).

Weaker granules have the structure which promotes dispersion of the active component, as they will break down as they pass through an inhaler.

Drying the granules can be performed in various manners. In general, it was found that the quicker the drying operation, the weaker the granules. Suitable drying means are for example formed by an oven. Especially preferred is drying while the granules are kept in motion, such as in a fluid bed dryer.

The particle size of the granules that (alone or in combination with some other vehicle) form the excipient lies between 50-1000 μm. Preferably, the particle size of the granules lies between 200-500 μm. The primary particle median geometric size of the granules lies in the range 1-170 μm, preferably in the range 1-15 μm.

The primary carrier material can be selected from a wide variety of materials which are preferably known to be suitable for DPI, including monosaccharides, such as glucose, fructose, mannose; polyols derived from these monosaccharides, such as sorbitol, mannitol or their monohydrates; disaccharides, such as lactose, maltose, sucrose, polyols derived from these disaccharides, such as lactitol, mannitol, or their monohydrates; oligo or polysaccharides, such as dextrins and starches.

Preferably the primary carrier material is a crystalline sugar such as glucose, lactose, fructose, mannitol or sucrose because such sugars are both inactive and safe. Most preferably, lactose is used.

The invention furthermore relates to a dry powder inhalation formulation which contains a pharmacologically active component and an excipient as claimed for delivery of the active component to the lungs.

The active component is for example selected from the group consisting of steroids, bronchodilators, cromoglycate, proteins, peptides and mucolytics, or from the group consisting of hypnotics, sedatives, analgesics, anti-inflammatory agents, anti-histamines, anti-convulscents, muscle relaxants, anti-spasmodics, anti-bacterials, antibiotics, cardiovascular agents, hypoglycaemic agents.

According to a further aspect thereof, the invention relates to a method for producing an excipient as claimed, comprising granulating a primary carrier material in a fluid binding agent and drying the granules thus obtained. The same preferred process parameters apply as indicated above.

The invention in a preferred embodiment thereof relates to lactose granules for use in dry powder inhalation preparations, which granules break down during inhalation in such a manner that they give a concentration of primary carrier material at stage 2 of the twin stage impinger determined by the antrone reaction of at least 5%, preferably at least 10%, more preferably at least 20%. These granules are obtainable by granulating lactose in a lactose solution or a solvent, such as ethanol, and drying the granules thus obtained. The active component is added to the finished granules.

The present invention is further illustrated in the example that follows.

### EXAMPLE

To demonstrate the concept of the present invention, granules with a particle size distribution of 200-500 μm were produced from α-lactose monohydrate (DMV International, the Netherlands) with a particle size distribution of 2-16 μm. A medium shear mixer (Kenwood) was used to granulate 450 g of lactose using an aqueous lactose solution, water or ethanol as the binding agent, added using a peristaltic pump (LKB). The mass was passed through a 1 mm screen (Erweka) and then dried in a fluid bed dryer (Aeromatic) or tray oven (Heraeus). The 200-500 μm fraction was prepared by screening a sieve shaker (Retsch).

The batches were as summarized in Table 1.

TABLE 1

| | Quantity | Lactose | | |
|---|---|---|---|---|
| | fluid | concentration | | |
| | binding | in fluid | Mixing | |
| | agent | binding agent | time | |
| Batch | (w/w) | (w/w) | (minutes) | Drying |
| 1 | 14% | 5% | 4 | Oven |
| 2 | 14% | 5% | 4 | Fluid bed |
| 3 | 14% | 5% | 3 | Fluid bed |
| 4 | 29% | Ethanol | 5 | Oven |
| | | (no lactose) | | |
| 5 | 20% | 5% | 4 | Fluid bed |
| 6 | 14% | 0% | 3 | Fluid bed |
| 7 | 14% | 50% | 3 | Fluid bed |

Determining the quantity of lactose on stage 2 by means of the antrone test is performed as follows. The antrone solution is prepared by dissolving 200 mg antrone in 200 g sulphuric acid. 1 ml of sample deposited at stage 2 of the impinger is collected and added to 2 ml of antrone solution. This mixture is allowed to stand for one hour. Subsequently the UV absorbance at 625 nm is determined. The result is given in the following table. The fine particle fraction (FPF) is the active component (e.g. the drug) reaching stage 2 (Table 2), determined as described hereinbelow.

TABLE 2
% Fine Particle Fraction (% FPF) represented by stage 2

| | % lactose | % Fine Particle |
|---|---|---|
| Batch no. | stage 2 | Fraction (% FPF) |
| 1 | 1 | 29.1 |
| 2 | 5 | 45.8 |
| 3 | 9 | 51.6 |
| 4 | 24 | 61.0 |
| 5 | 2 | 38.6 |
| 6 | 6 | 48.1 |
| 7 | 8 | 50.1 |
| Reference | 0 | 31.2 |
| (DCL 15) | | |

The granules were blended with the drug sodium cromoglycate (1.8% (w/w)). On completion of the mixing process it was clearly evident that the granules had maintained their initial shape. The formulations were assessed in vitro using the twin stage impinger at 60 1/min which has a cut off diameter of 6.4 μm, using the Novolizer Inhaler (Sofotec). The amount of active component on each stage was determined using UV spectroscopy. (Table 3).

TABLE 3

| | | | Stage 2 | |
|---|---|---|---|---|
| | Inhaler | Stage 1 | % active | |
| | % active | % active | component | CU (%) |
| Batch | component | component | (=FPF) | (% RSD) |
| 1 | 13.5 | 66.7 | 29.1 | 97.7 (6.8) |
| 2 | 13.7 | 51.8 | 45.8 | 92.0 (6.6) |
| 3 | 8.8 | 40.3 | 51.6 | 96.2 (2.7) |
| 4 | 7.7 | 23.8 | 61.0 | 97.8 (3.4) |
| 5 | 16.8 | 50.2 | 38.6 | 94.6 (6.7) |
| 6 | 10 | 43.4 | 48.1 | 96.2 (4.4) |
| 7 | 9.5 | 36.9 | 50.1 | 97.7 (1.7) |
| Reference | 15.8 | 59.7 | 31.2 | 97.2 (4.6) |

Table 3 shows the in vitro deposition values for the 8 batches of granules (7 according to the invention and 1 reference (DCL 15 from DMV International, the Netherlands)), detailing recovery of active component from the inhaler, stage 1, stage 2 (FPF), content uniformity (CU) and relative standard deviation (% RSD)

Granulation is determined distribution of liquid over the surface of particles, forming liquid bridges between particles. This is followed by the evaporation of the liquid resulting in the formation of solid bridges which binds particles together forming granules.

From the results of this experiment it can de derived that decreasing the amount of liquid available for dispersion in granulation, reduces the amount of potential solid bridges producing weaker granules (batch nos. 5 and 2).

Poor dispersion of liquid as a result of shorter mixing times does also produce weaker granules (batch nos. 2 and 3).

Furthermore, it was found that the slower the drying. rate the larger the crystals, formed during recrystallisation (batch nos. 1, 2 and 4). Fluid bed drying is faster.

Solids concentration in the liquid has no effect due to the relatively good solubility of lactose (batch nos. 3, 6 and 7).

1-26. (canceled)

28. Excipient for dry powder inhalation preparations comprising granules made of primary carrier material, which granules break down during inhalation in such a manner that they give a concentration of primary carrier material at stage 2 of the twin stage impinger of at least 5%, which excipient is obtainable by granulating a primary carrier material in a fluid binding agent and drying the granules thus obtained.

29. Excipient as claimed in claim 28, wherein the concentration of primary carrier material at stage 2 of the twin stage impinger is at least 10%.

30. Excipient as claimed in claim 28, wherein the concentration of primary carrier material at stage 2 of the twin stage impinger is at least 20%.

31. Excipient as claimed in claim 28, wherein the fluid binding agent is an aqueous solution of the primary carrier material.

32. Excipient as claimed in claim 28, wherein the fluid binding agent is a solvent, in particular ethanol.

33. Excipient as claimed in claim 28, wherein the fluid binding agent is water.

34. Excipient as claimed in claim 28, wherein the drying is performed in an oven.

35. Excipient as claimed in claim 28, wherein the drying is performed while the granules are kept in motion, such as in a fluid bed dryer.

36. Excipient according to claim 28, wherein the particle size of the granules lies between 50-1000 μm.

37. Excipient according to claim 28, wherein the particle size of the granules lies between 200-500 μm.

38. Excipient according to claim 28, wherein the primary particle median geometric size of the granules lies in the range 1-170 μm.

39. Excipient according to claim 28, wherein the primary particle size median geometric size of the granules lies in the range 1-15 μm.

40. Excipient according to claim 28, wherein the primary carrier material is a monosaccharide, such as glucose, fructose, mannose; a polyol derived from these monosaccharides, such a sorbitol, mannitol or their monohydrates; a disaccharide, such as lactose, maltose, sucrose, polyol derived from these disaccharides, such as lactitol, mannitol, or their monohydrates; an oligo or polysaccharide, such as dextrins and starches.

41. Excipient according to claim 28, wherein the primary carrier material is a crystalline sugar such as glucose, lactose, fructose, mannitol or sucrose.

42. Excipient according to claim 28, wherein the primary carrier material of the granules is lactose.

43. A dry powder inhalation formulation which contains a pharmacologically active component and an excipient according to claim 28, for delivery of the active component to the lungs.

44. A dry powder inhalation formulation according to claim 43, in which the active component is selected from the group consisting of sterioids, bronchodilators, cromoglycate, proteins, peptides and mucolytics.

45. A dry powder inhalation formulation according to claim 43, in which the active component is selected from the group consisting of hypnotics, sedatives, analgesics, anti-inflammatory agents, anti-histamines, anti-convulsants, muscle relaxants, anti-spasmodics, anti-bacterials, anti-biotics, cardiovascular agents and hypoglycaemic agents.

46. Method for producing an excipient as claimed in claim 28, comprising granulating a primary carrier material in a fluid binding agent and drying the granules thus obtained.

47. Method as claimed in claim 46, wherein the fluid binding agent is an aqueous solution of the primary carrier material.

48. Method as claimed in claim 46, wherein the fluid binding agent is a solvent, in particular ethanol.

49. Method as claimed in claim 46, wherein the fluid binding agent is water.

50. Method as claimed in claim 46, wherein the drying is performed in an oven.

51. Method as claimed in claim 28, wherein the drying is performed while the granules are kept in motion, such as in a fluid bed dryer.

52. Lactose granules for use in dry powder inhalation preparations, wherein the granules break down during inhalations in such a manner that they give a concentration of primary carrier material at stage 2 of the twin stage inpinger of at least 5%.

53. Lactose granules according to claim 52, wherein the granules break down during inhalation in a manner that they give a concentration of primary carrier material at stage 2 of the twin stage impinger of at least 10%.

54. Lactose granules according to claim 52, wherein the granules break down during inhalation in a manner that they give a concentration of primary carrier material at stage 2 of the twin stage inpinger of at least 20%.

55. Use of an excipient as claimed in claim 46 for the preparation of a dry powder inhalation preparation for the treatment of diseases of the respiratory tract.
